(19) United States
(12) Patent Application Publication
(10) Pub. No.: US 2009/0311767 A1
(43) Pub. Date: Dec. 17, 2009

(54) Method for molecular delivery into cells using naonotube spearing

(76) Inventor: Thomas C. Chiles, Norfolk, MA (US)

Correspondence Address: GREENBERG TRAURIG, LLP, ONE INTERNATIONAL PLACE, 20th FL, ATTN: PATENT ADMINISTRATOR, BOSTON, MA, 02110, US

(21) Appl. No.: 11/919,025

(22) PCT Filed: Apr. 21, 2006
(86) PCT No.: PCT/US2006/015061

§ 371 Date: Oct. 19, 2007

Related U.S. Application Data
(60) Provisional application No. 60/674,120, filed on Apr. 21, 2005.

(51) Int. Cl. C12N 13/00

(52) U.S. Cl. 435/173.4; 977/906

(57) ABSTRACT

The present invention discloses a method of delivering a macromolecule to a cell, comprising engaging the macromolecule to a nanotube wherein the nanotube comprises a magnetic particle, placing the nanotubes into a sample wherein the sample comprises a plurality of cells and applying a force to the sample wherein the force causes the nanotubes to collide with the cells and thereby spear the cells. The method further discloses applying a second force which drives the nanotubes into the cell. Furthermore, the invention discloses a method of transfecting and/or transducing a cell, comprising engaging a macromolecule to a nanotube, placing the nanotubes into communication with the cells and applying a force to the nanotubes which causes the nanotubes to collide with the cells and thereby pierce the cellular membrane. Once inside the cell, the macromolecule may disengage from the nanotube.

### RELATED APPLICATIONS

This application claims the benefit of U.S. Provisional Application Ser. No. 60/674,120, filed Apr. 21, 2005, the entirety of which is hereby incorporated herein by reference for the teachings therein.

### GOVERNMENT SUPPORT

The invention was supported, in whole or in part, by the U.S. Department of Energy (DE-FG02-00ER45805), the National Science Foundation (NIRT 0304506), and the National Institutes of Health (AI-34586). The Government has certain rights in the invention.

### BACKGROUND

A problem with macromolecules and/or drug delivery by conventional methods is the problem of low efficiency as it pertains to delivery of materials into primary (non-transformed) cells and tissues. Typically a low percentage of cells actually take up the material (about 3% to about 20%). In addition, the conventional techniques are associated with a high percentage of cell death.

As such, there is a need in the art for a method allowing for highly efficient macromolecule delivery with high viability which may be used with hard-to-transfect cells, including B cells and primary neurons.

Methods for molecular delivery into cells using nanotube spearing are disclosed herein.

According to aspects illustrated herein, there is provided a method of delivering a macromolecule to a B cell comprising: engaging a macromolecule to at least one nanotube wherein the nanotube comprises a magnetic particle; placing the nanotubes into a sample wherein the sample comprises a plurality of cells; applying a force to the sample wherein the force causes the nanotubes to collide with the cells to spear the cells; and applying a second force which drives the nanotubes into the cell.

According to aspects illustrated herein, there is provided a method of transfecting a B cell comprising: engaging a macromolecule to at least one nanotube wherein the nanotube comprises a magnetic particle; placing the nanotubes into a sample wherein the sample comprises a plurality of cells; applying a force to the sample wherein the force causes the nanotubes to collide with the cells to spear the cells; applying a second force which drives the nanotubes into the cells; and disengaging the macromolecule from the nanotube.

According to aspects illustrated herein, there is provided a method of transducing a B cell comprising: engaging a macromolecule to a nanotube wherein the nanotube comprises a magnetic particle; placing the nanotubes into communication with a plurality of cells; applying a force to the nanotubes which causes the nanotubes to collide with the cells and pierce the cellular membrane; and disengaging the macromolecule from the nanotube.

### BRIEF DESCRIPTION OF THE DRAWINGS

The presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments.

FIG. 1 shows a presently disclosed embodiment of a method of molecular delivery into cells using nanotube spearing comprising a two-step process.

FIG. 2A shows cells without nanotube spearing. FIG. 2B shows cells with nanotube spearing.

FIG. 3A shows transduced cells after spearing by nanotubes with pEGFP-c1. FIG. 3B shows a control. FIG. 3C shows charts and graphs illustrating cell viability following nanotube spearing.

FIG. 4A shows EGFP transduction in primary cultured cells by nanotube spearing with a (10,10) spearing protocol. FIG. 4B shows EGFP transduction in primary cultured cells by nanotube spearing with a (20,20) spearing protocol. FIG. 4C shows EGFP transduction in primary cultured mouse cortical neurons 48 hours after spearing by spearing protocol (20,20).

FIG. 5A shows a scanning electron microscope (SEM) image of drivable nanotubes on a substrate. The inset of FIG. 5A shows a corresponding transmission electron microscope (TEM) image. FIG. 5B shows an SEM image of magnetically non-drivable nanotubes on a substrate. The inset of FIG. 5B shows a corresponding TEM image.

FIG. 6A shows cell viability after nanotube spearing for MCF-7 cells. FIG. 6B shows cell viability after nanotube spearing for primary B cells.

FIG. 7 shows an embodiment of a tip of a carbon nanotube comprising a nickel particle.

FIG. 8 shows an embodiment of a nanotube wherein the nickel particle has been removed.

FIG. 9 shows EGFP transfection with Lipofectamine 2000 in Bal17 cells.

FIG. 10 shows EGFP transfection with Lipofectamine 2000 in primary B cells.

FIG. 11A shows nuclei staining with DAPI in cortex neurons for a control. FIG. 11B shows nuclei staining with DAPI in cortex neurons for a speared cell.

While the above-identified drawings set forth preferred embodiments, other embodiments are also contemplated, as noted in the discussion. This disclosure presents illustrative embodiments by way of representation and not limitation. Numerous other modifications and embodiments can be devised by those skilled in the art which fall within the scope and spint of the principles of the presently disclosed embodiments.

### DETAILED DESCRIPTION

Methods for molecular delivery into cells using nanotube spearing are disclosed herein.

As used herein, the terms “B cell” or “B cells” refer to lymphocytes that play a key role in the humoral immune response. The human body produces millions of B lymphocytes from the bone marrow on a daily basis. Each B cell “clone” normally expresses on its surface an immunoglobulin (Ig) protein (referred to as a B cell receptor, or the BCR) and also secretes this Ig in response to foreign substances. B lymphocytes are secrete cytokines (proteins with immunomodulatory properties) that, in turn regulate subsequent immune responses. In general, B cells derived from the bone marrow as part of the normal production of B cell in the body are refereed to as primary B cells. Immunologists typically study primary B cells in the laboratory by first purifying the B cells from mice or human sources and then placing the primary B cells in tissue culture (referred to herein as ex vivo). By contrast, lymphomas (such as the mouse Bal17 B lymphoma cited herein) and leukemic B cells result from transformation and exhibit tumor or tumor-like properties.

Vertically aligned carbon nanotubes grown by plasma enhanced chemical vapor deposition (“PECVD”) have ferromagnetic catalyst nickel (“Ni”) particles enclosed in their tips. This structure renders nanotubes a useful response to magnetic agitations. Such a property can be simply demonstrated by placing a container of carbon nanotube suspension on a magnetic stir plate without a magnetic stirring bat in the container. If the nanotubes are magnetically drivable, a rotating “cloud” synchronizing with the magnetic field can be visually noticed. The momentum of the carbon nanotubes can be utilized to penetrate cell membranes, which is termed as nanotube spearing, thereby shuttle macromolecules immobilized on the carbon nanotubes into cells. Nanotube spearing should be more controllable than other nanotube-based molecular delivery approaches. Considering the nano-scale size, nanotube spearing are milder than the ballistic method to implement a mechanical macromolecule delivery with high cell viability.

In addition, biomineralization and other uptake by endocytosis are alternate approaches to transport plasmids across the plasma membrane. But before the post-uptake DNA plasmids reach the nucleus, their intracellular trafficking has to proceed via the endo- or lysosomal pathway, where a large fractional amount of the plasmids are hydrolyzed. The fates of the plasmids in this process will determine the transduction efficiency and contribute to the difference in transduction between primary and transformed cells. With nanotube spearing, the nucleus may also be penetrated, and, therefore, receive plasmid DNA directly from the invasive nanotubes. Such a gene delivery will straightforwardly enhance genetic transduction of exogenous DNAs, particularly, in primary cells.

Introduction of exogenous DNA into mammalian cells represents a powerful approach for manipulating signal transduction. However, the current available techniques have serious limits on either low transduction efficiency or low viability. The presently disclosed embodiments disclose a highly efficient molecular delivery technique, named nanotube spearing, based on the penetration on Ni-particle embedded nanotubes into cell membranes by magnetic field driving. In an embodiment, a macromolecule is immobilized onto a nanotube, and subsequently speared into a targeted cell. In an embodiment, a DNA plasmid encoding the enhanced green fluorescent protein (“EGFP”) sequence is immobilized onto a nanotube, and subsequently speared into a targeted cell. In an embodiment, a nucleic acid is immobilized onto a nanotube, and subsequently speared into a targeted cell. Those skilled in the art will recognize that various types of macromolecules may be delivered to cells via nanospearing and remain within the spirit and scope of the presently disclosed embodiments.

In an embodiment, a macromolecule is delivered to a cell. In an embodiment, the macromolecule is delivered to a mammalian cell. In an embodiment, a macromolecule is delivered to a Bal17 B-lymphoma cells. In an embodiment, a macromolecule is delivered to an ex vivo B cells. In an embodiment, a high transduction efficiency in primary neurons with high viability is disclosed. In an embodiment, transfection of cells which are difficult to transfect occurs. Those skilled in the art will recognize that various types of cells are within the spirit and scope of the presently disclosed embodiments. As such, the presently disclosed embodiments disclose a powerful method for high efficient gene transfer in a variety of cells, especially, the hard-to-transfect cells.

The presently disclosed embodiment provides a method wherein macromolecules may be delivered to a cell. In an embodiment, a macromolecule is engaged to a nanotube. In an embodiment, the macromolecule is engaged to the nanotube by a covalent bond. In an embodiment, the macromolecule is engaged to the nanotube by an ester bond. Those skilled in the art will recognize that various ways of engaging the macromolecule to the nanotube is within the spirit and scope of the presently disclosed embodiments.

In an embodiment, the macromolecule is engaged at the tip of the nanotube. In an embodiment, the macromolecule is engaged on the outside of the nanotube. In an embodiment, the macromolecule is engaged at a plurality of locations along the length of the nanotube. Those skilled in the art will recognize that the macromolecule may be located at a single location or a plurality of locations along the nanotube and remain within the spirit and scope of the presently disclosed embodiments.

In an embodiment, a single macromolecule is engaged to a nanotube. In an embodiment, a plurality of macromolecules are engaged to the nanotube. In an embodiment, only one type of macromolecule is engaged to the nanotube. In an embodiment, various types of macromolecules are engaged to the nanotube. Those skilled in the art will recognize that any number of macromolecules and any number of different types (i.e., various proteins, etc) of macromolecules may be engaged to a nanotube and remain within the spirit and scope of the presently disclosed embodiments.

In an embodiment, nanotube spearing is used to transfect/transduce cells which are difficult to transfect. In an embodiment, the method transfects MCF-7 cells. In an embodiment, the method transfects Bal17 cells. In an embodiment, the method transfects ex vivo cultures of murine B lymphocytes. In an embodiment, the method may be used to deliver materials to cells in vivo. Those skilled in the art will recognize that various cells are within the spirit and scope of the presently disclosed embodiments.

In an embodiment, the macromolecule disengages from the nanotube into the cytoplasm of the target cell. In an embodiment, the macromolecule may be targeted for a particular cellular component. Those skilled in the art will recognize there are various ways to target a macromolecule for a specific cellular component; all such ways are within the spirit and scope of the presently disclosed embodiments.

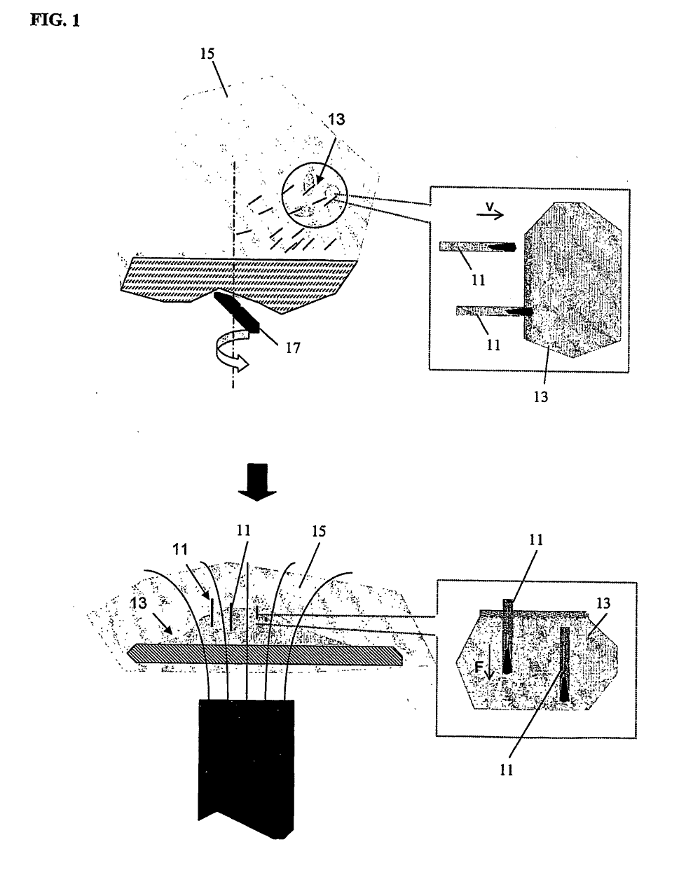

FIG. 1 shows a two-step procedure for a method of nanotube spearing of the presently disclosed embodiments. The upper portion of FIG. 1 shows the first step of the two-step procedure. In step one, a solution 15 is placed on a substrate wherein the solution 15 contains a plurality of cells 13 and a plurality of nanotubes 11. As shown, a rotating magnetic field drives a plurality of nanotubes 11 to spear a plurality of cells 13 on a substrate. As such, the rotating magnetic field provided by a magnet 17 causes the nanotubes 11 to “collide” with the plurality of cells 13. As such, a plurality of nanotubes 11 “spear” a plurality of cells 13. The inset at the top of FIG. 1 shows a velocity profile (v) of a nanotube 11 heading towards a cell 13. Those skilled in the art will recognize that various methods of creating collisions between a plurality of nanotubes 11 and a plurality of cells 13 are within the spirit and scope of the presently disclosed embodiments.

The bottom of FIG. 1 shows the second step of the two-step procedure. At the beginning of step two, a plurality of nanotubes 11 are positioned in the various cell 13 membranes. In an embodiment, step two comprises driving the nanotube 11 through the cellular membrane and into the cell. In an embodiment, a static field persistently pulls nanotubes into the plurality of cells. The inset at the bottom of FIG. 1 shows the force profile of a nanotube 11 being driven into the target cell 13. Those skilled in the art will recognize any mechanism to drive the nanotube 11 through the membrane and into the cytoplasm is within the spirit and scope of the presently disclosed embodiments. Once in the cell 13, a macromolecule engaged to the nanotube may become dissociated from the nanotube and enter the cytoplasm of the cell. Those skilled in the art will recognize that the macromolecule may become dissociated from the nanotube in any of a variety of ways and remain within the spirit and scope of the presently disclosed embodiments.

As such, in an embodiment, the method of the presently disclosed embodiments comprises a first step and a second step. In an embodiment, step one lasts for an amount of time designated as time period one (“a hours”). In an embodiment, step two lasts for an amount of time designated as time period two.(“b hours”). Throughout this specification, these respective time periods are indicated as “spearing protocols and are shown numerically as (a,b). For example, a spearing protocol which undergoes step one for 10 hours and step two for 5 hours will be represented as spearing protocol (10,5). Table 1 below shows experimental results for various spearing protocols.

TABLE 1
Summary of results for nanotube spearing

| | | Nanotubes | | | | |
|---|---|---|---|---|---|---|
| | | with Ni | without Ni | | pcDNA3.1 | |
| Treatment | Cell type | particle | particle | pEGFP-c1 | vector | Result |
| Spearing (3, 7) | Bal17 | yes | no | yes | no | fluo |
| Spearing (3, 7) | Bal17 | yes | no | no | no | non-fluo |
| Spearing (3, 7) | Bal17 | no | no | yes | no | non-fluo |
| Spearing (3, 7) | Bal17 | no | yes | yes | no | non-fluo |
| Spearing (15, 0) | Bal17 | yes | no | yes | no | weak fluo |
| Spearing (0, 15) | Bal17 | yes | no | yes | no | weak fluo |
| Incubation | Bal17 | yes | no | yes | no | nearly |
| | | | | | | background |
| Spearing | Primary B | yes | no | yes | no | fluo(20, 20) |
| (10, 10), (20, 20) | cells | | | | | |
| Spearing | Primary | yes | no | yes | no | fluo(20, 20) |
| (10, 10), (15, 15), | neurons | | | | | |
| (20, 20) | | | | | | |

In Table 1, “fluo” stands for fluorescence signal and “non-fluo” stands for fluorescence signal.

In an embodiment, the nanotubes are induced to collide with cells and the nanotubes are driven into the cytoplasm in a single step. Those skilled in the art will recognize that any process of inducing nanotubes engaged to macromolecules to collide with a plurality of target cells and deliver the macromolecule to the cell is within the spirit and scope of the presently disclosed embodiments.

In an embodiment, the macromolecule engaged to the nanotube comprises a capability to seek out specific cellular compartments once dissociated from the nanotube. In an embodiment, the macromolecule comprises a tag in order to observe the macromolecule once the macromolecule has become dissociated from the nanotube. In an embodiment, the tag is a fluorescent tag. Those skilled in the art will recognize that various tags are with the spirit and scope of the presently disclosed embodiments.

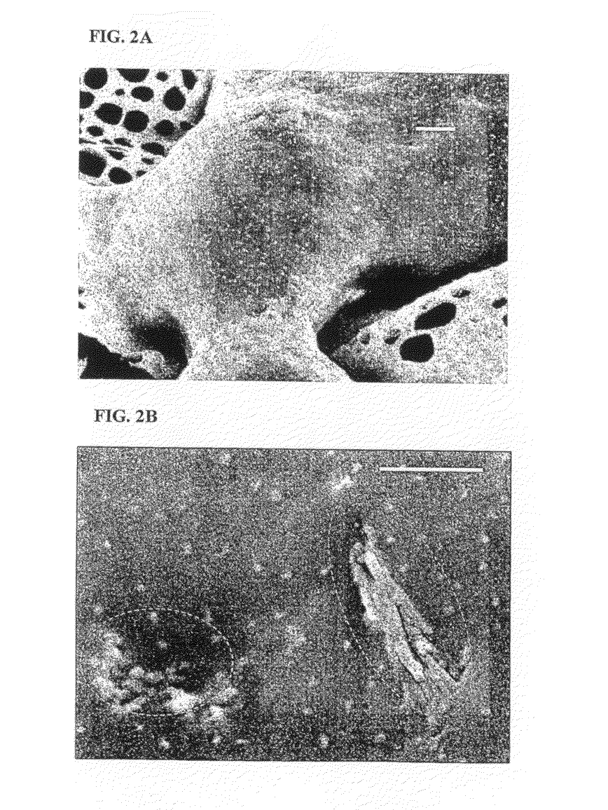

FIG. 2A and FIG. 2B show nanotube spearing in MCF-7 cells. In an embodiment, the cells were cultured on a grid. FIG. 2A shows images of cell membranes without nanotube spearing. FIG. 2B shows images of cells with nanotube spearing. Scale bars in FIG. 2A and FIG. 2B are 1 μm and 500 nm respectively. Dashed ovals in FIG. 2B mark the nanotubes in the membrane. In an embodiment, the microvilli in the membranes of the cells shown in FIG. 2A and FIG. 2B have the same site density, which is about 15/μm².

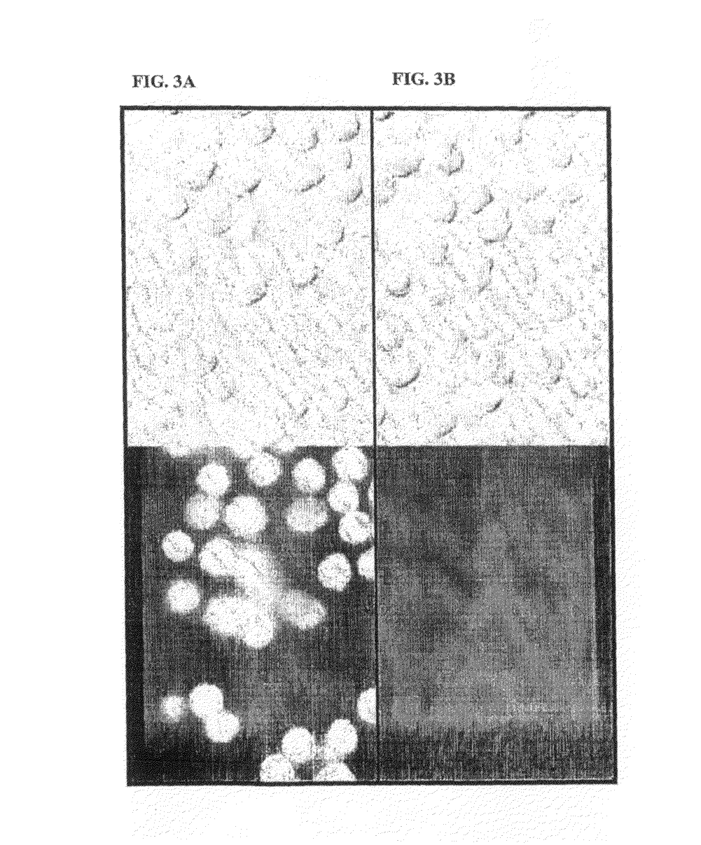

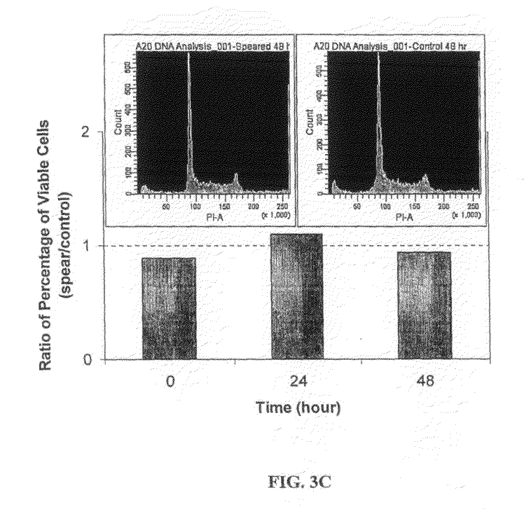

FIG. 3A, FIG. 3B and FIG. 3C show transduction of EGFP in Bal17 cells by the presently disclosed methods of nanospearing. FIG. 3A shows the transduced cells after spearing by nanotubes with pEGFP-c1 plasmids. FIG. 3B shows the control, in which cells were speared by nanotubes with empty pcDNA3.1 vector. In an embodiment, the spearing protocol used was (3,7). The top panel of FIG. 3A and FIG. 3B is the bright field view and the bottom panel of these figures is the dark field view. Scale bars indicate 10 μm.

FIG. 3C shows a table illustrating the viability of the speared cells as compared to the control cells. Control and nanotube speared Bal17 cells were cultured for various times after spearing (0, 24, and 48 hours). Cells were stained with propidium iodide and viability assessed by flow cytometry. FIG. 3C shows the resulting data represented as a ratio of the percentage of viable cells (spearing/control) with a total of about 5,000 cells analyzed. In the inset of FIG. 3C, control and nanotube speared Bal17 cells were cultured for about 48 hours after which cell cycle analysis Was determined by propidium iodide staining followed by flow cytometric analysis of DNA content. Individual histograms for the control and speared Bal17 cell population are shown. The percentage of cells in G₀/G₁, S, G₂/M for control cells corresponded to 44.8, 47.5, and 7.7, respectively, whereas for nanotube speared cells the percentage of G₀/G₁, S, G₂/M corresponded to 42.6, 49.7, and 7.7, respectively.

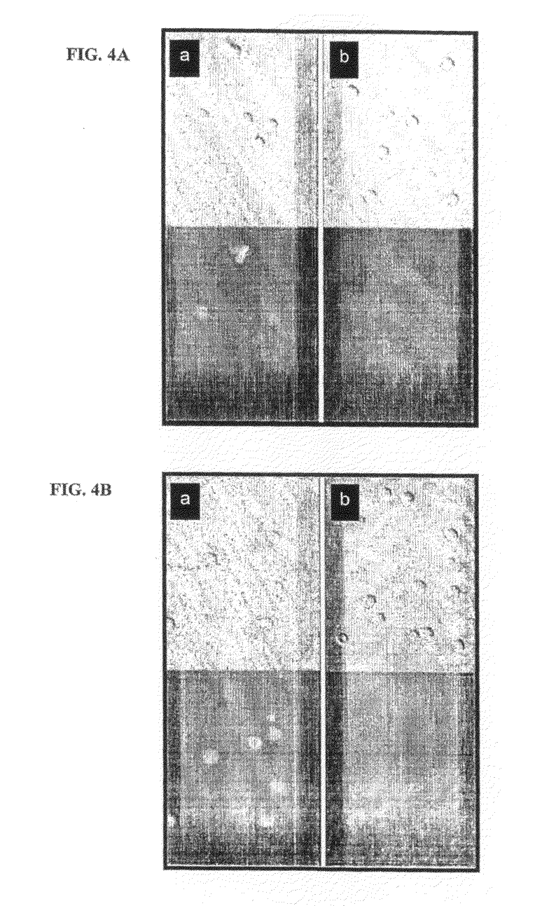

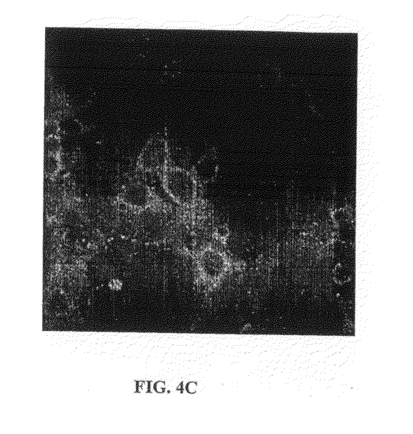

FIG. 4A, FIG. 4B, and FIG. 4C show EGFP transduction in primary cultured cells by nanotube spearing. FIG. 4A shows primary B cells speared with spearing protocol (10,10). FIG. 4B shows primary B cells speared with spearing protocol (20,20). In both FIG. 4A and FIG. 4B, the top panels are bright fields, and the bottom panels are dark fields. FIG. 4C shows EGFP transduction in primary cultured mouse cortical neurons 48 hours after spearing by spearing protocol (20,20). Scale bars indicate 10 μm.

### EXAMPLES

The following examples are merely illustrative of the presently disclosed embodiments. As such, these examples in no way limit the scope of the presently disclosed embodiments and are merely representative of various aspects and features of the presently disclosed embodiments.

### Responses of Nanotubes to Magnetic Agitation:

In an embodiment, the method utilizes nanotubes wherein the nanotubes are responsive to a magnetic agitation. In an embodiment, the magnetic agitation helps to drive the nanotubes to a desired location for macromolecule delivery. An electron microscopy observation revealed that the magnetically drivable and non-drivable carbon nanotubes are morphologically different. Drivable carbon nanotubes are short (less than about 2 μm) and have a magnetic particle with an aspect ratio, defined as the ratio of a length to a width, of about 2.9, while a non-drivable carbon nanotubes are longer (greater than about 15 μm) and have an aspect ratio of about 0.7.

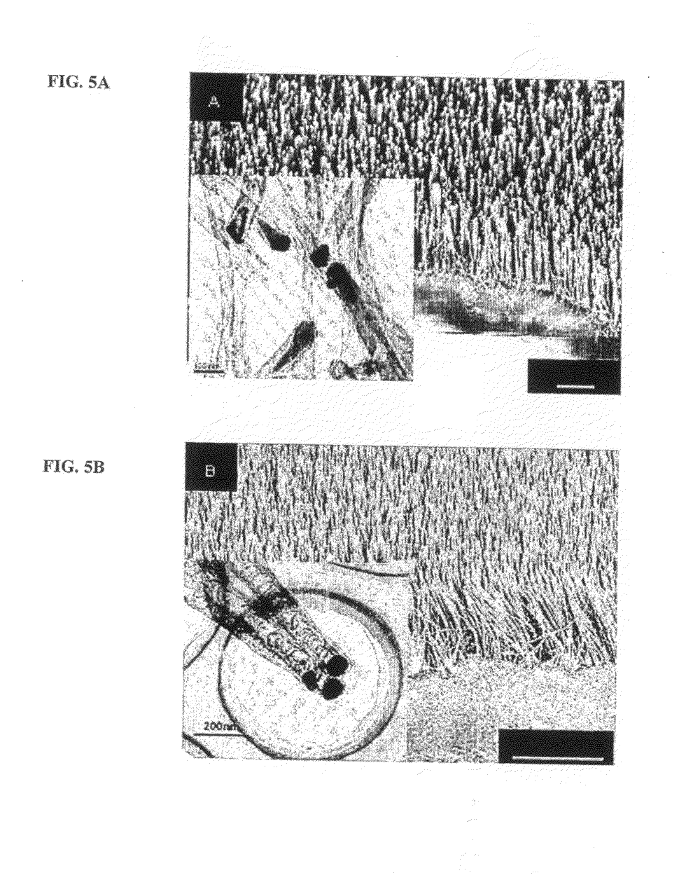

FIG. 5A and FIG. 5B show an embodiment of a carbon nanotube and Ni particle morphology. FIG. 5A shows an SEM image of a plurality of magnetically drivable nanotubes on a substrate. As shown, in an embodiment, the length of the nanotubes is about 1.5 μm (scale bar is 1 μm). The inset of FIG. 5A shows a corresponding transmission electron microscope (TEM) image of the elongated nanowire-like Ni particles embedded at the tips of the nanotubes. FIG. 5B shows an SEM image of magnetically non-drivable nanotubes on the substrate. In an embodiment, the magnetically non-drivable nanotubes are about 15 μm long (scale bar is 10 μm). The inset of FIG. 5B shows a corresponding TEM image shows with the Ni particles embedded at the tips have prolate shape. The particle aspect ratios are about 2.9±0.13 and 0.7±0.04 for magnetically drivable and non-drivable tubes, respectively (all samples were tilted 45° for SEM imaging).

It is of note that PECVD regardless the length of the nanotubes can be well dispersed in ethyl alcohol without using a surfactant. Nevertheless, in a separate experiment, about 8 mg/ml Nanosperse AQ (NanoLab, Inc.) was used. Nanosperse AQ is able to keep about 100 mg/10 ml nanotubes stably in water suspension for weeks, to suspend the nanotubes. Ideal suspension of both kinds of nanotubes in water were secured so that the behaviors of the nanotubes in magnetic fields were independent to their dispersibility. The same results were obtained with or without the use of Nanosperse AQ.

### Membrane Penetration by Nanotube Spearing:

The spearing of the cell membranes with carbon nanotubes was first demonstrated in MCF-7 cells. The spearing setup and procedure is illustrated in FIG. 1. The magnetic field drives the nanotubes in a medium towards cells cultured on a dish. After the preliminary spearing by rotating field, the cells were transferred to culture dishes containing nanotube-free medium for enhancing the spearing by a static field of a permanent magnet.

FIG. 2A shows typical membranes of cells without nanotube spearing. FIG. 2B shows typical membranes of cells with nanotube spearing. In FIG. 2B, a portion of the nanotube which is still outside the membrane is visible.

A comparison was performed between cell membranes on the SEM images of speared cells exposed to various combinations of the spearing conditions. A process with about a 3 minute rotating field plus about a 7 minute static field (expressed as “(3,7)”), resulted in more nanotube-embedding in a membrane (approximately 76%) than the (10,0) or (0,10) combinations (which resulted in lower than about 10%). In addition, greater than about 90% of MCF-7 cells were viable after the rotating plus static field spearing; these results were obtained with the use of trypan blue staining.

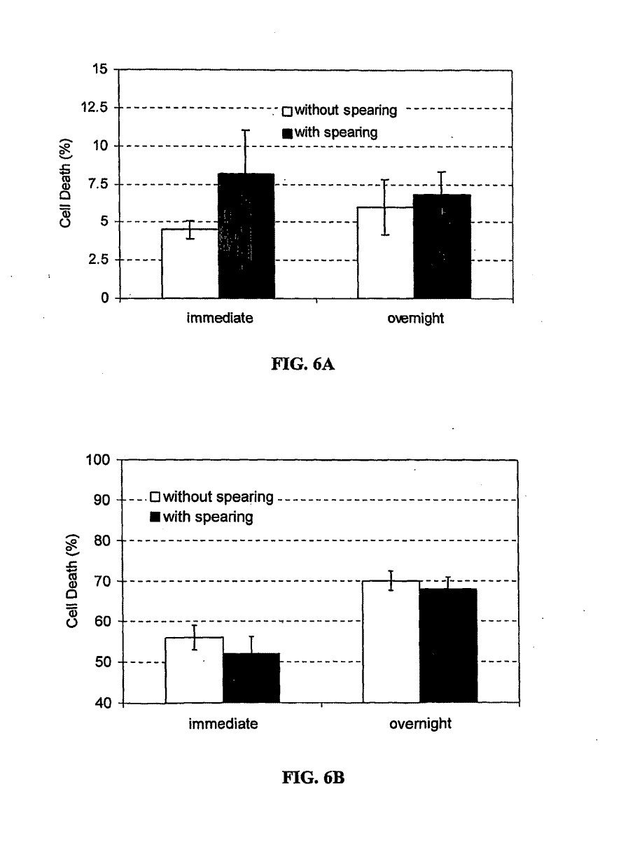

FIG. 6A and FIG. 6B display results wherein cell death without spearing is compared to cell death with spearing. The results show the high viability of cells which have undergone nanospearing. FIG. 6A shows cell mortality after nanotube spearing in MCF-7 cells. FIG. 6B shows cell mortality after nanotube spearing in primary B cells. The spearing protocol in MCF-7 cells and primary B cells are (3,7) and (20,20) respectively. The cells without spearing experienced the whole procedure without nanotubes in the medium (error bars are standard errors of the means).

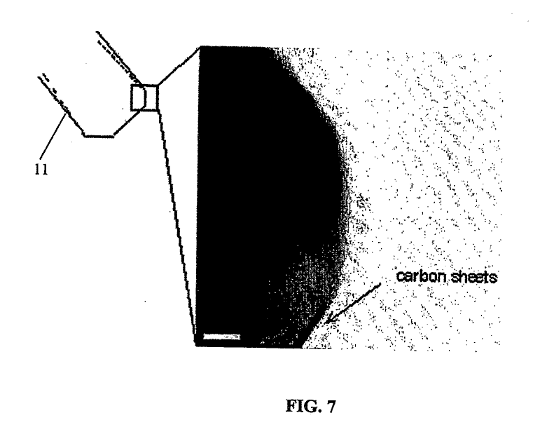

A concern with carbon nanotube spearing is the intracellular Ni²⁺ contamination due to the possible incomplete encapsulation of the magnetic particles in the nanotubes. This may alter the cellular signal transduction including gene expression. As shown in FIG. 7, high resolution TEM study showed that the Ni particles were enclosed in the nanotubes by layers of graphene sheets and amorphous carbon (scale bar is 5 nm). The layers of graphene sheets and/or amorphous carbon render biocompatibility of the nanotubes by keeping the Ni particle separated from the cytoplasma. As such, the Ni particles are separated from the cytoplasm and Ni contamination is avoided.

### DNA Delivery by Nanotube Spearing in Dividing Mammalian Cells:

Since membrane penetration was strongly indicated by the above data, the next step was to spear mammalian cells and deliver macromolecules. For these experiments, a mammalian expression vector (pEGFP-c1) containing EGFP sequence was immobilized on the nanotubes. A pcDNA3.1 vector devoid of gene insert was used as a negative control. The intracellular delivery of plasmid DNA by nanotube spearing was first carried out in Bal17 cells. Following nanotube spearing, Bal17 cells were cultured for about 24 hours. Fluorescence microscopy was used to examine EGFP expression. As shown in FIG. 3A and FIG. 3B, approximately 100% of Bal17 cells exhibit fluorescence signal following nanotube spearing with pEGFP-c1 (FIG. 3A), where no detectable fluorescence was observed in parallel control Bal17 cells (FIG. 3B). Flow cytometry revealed approximately 85% of the cells were EGFP^{hi}. The viability of Bal17 cells before and after spearing was also analyzed with a flow cytometer. As shown in FIG. 3C, no noticeable difference between the cell viability of control and transduced cells at 0, 24, and 48 hours after the spearing. In addition, the nanotube spearing did not change the cell cycle based on the comparison to control Bal17 cells as determined by propidium iodide staining of nuclei.

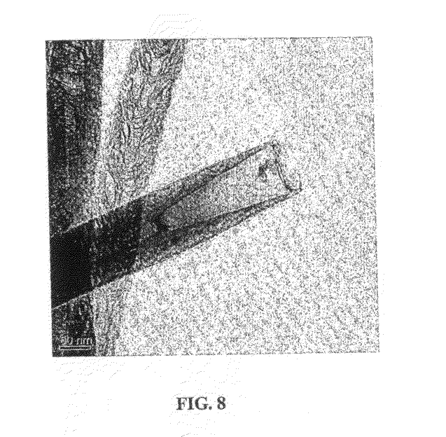

Various conditions were tested in order to understand mechanisms of nanotube spearing. The results of these tests are summarized in Table 1. FIG. 8 shows a Ni particle deprived nanotube. As shown in FIG. 8, extreme oxidative conditions were employed to remove the Ni particles in the nanotubes (a 9:1 concentrated H₂SO₄/30% H₂O₂ solution (Zhao, W. et al. J.A.C.S 124, 12418-19 (2002)) was used to etch off the graphene sheets and subsequently dissolve the encapsulated Ni particles). A spearing protocol (3,7) with such Ni deprived nanotubes yields no fluorescence signal in the cells. Also, the incubation of the cells with normal plasmids immobilized nanotubes did not produce any transduction. Additionally, no signal was yielded by the magnetic treatment to cells with the presence of DNA plasmids only. These results indicated that the magnetic actuation of the nanotubes was indispensable obtain gene expression. With these experimental conditions, the uptake pathway was not one of the transducing mechanisms. The effectiveness of each of the two spearing steps was evaluated in term of the transduction by two protocols, i.e. (15,0) and (0,15). Both protocols produced low levels of fluorescence signals barely above the background. As such, the results indicated that a two-step protocol helps lead to an efficient transduction.

### DNA Delivery by Nanospearing in Non-Dividing Mammalian Cells:

With the use of the present method, nanotube spearing provides an improvement in the transduction in primary cells. The ex vivo splenic B cells and primary culture of cortical neurons were used for the experiment. Murine splenic B cells were purified from Balb/c mice and cultured. The cortical neurons were separated from embryonic C57/BL6 mouse. As shown in FIG. 4A and FIG. 4B, approximately 100% of the primary B cells expressed EGFP about 24 hour after the nanotube spearing as determined by fluorescence microscopy. In addition, by comparing FIG. 4A with FIG. 4B, it may be observed that fluorescence levels may be increased with the spearing time. Remarkably, even with the longest spearing periods (20,20), there was no obvious decrease in cell viability as compared to the samples without spearing (see FIG. 6B), in which the cells went through the same spearing procedures in nanotube free medium. The cell death observed with primary B cells is due to the absence of fetal calf serum (FCS) and the B-cell survival factors, including but not limited to interleukin-4 (IL-4), in the medium before and during the spearing, and is subjected to further optimization.

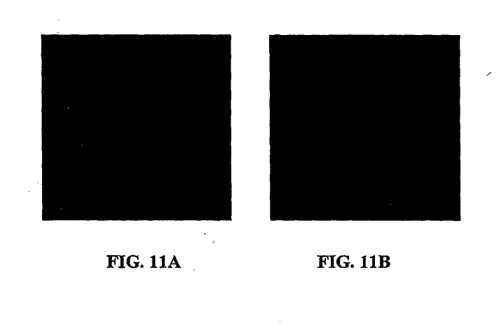

Additionally, experiments were carried out at spearing protocols (10,10), (15,15), and (20,20) in primary cortical neurons for EGFP transduction. No fluorescence was found in 24 hours. After 48 hours, the neuron turned green in the (20,20) group (as seen in FIG. 4C). As determined by fluorescence microscopy, the percentage of green cells was about 80%. Process retraction was noticed in the neurons of all spearing groups in about 24 hours. However, they returned to normal in 24 hours. Nuclei staining with about 0.5 μg/ml 4,6-diamidino-2-phenylindole (DAPI) showed a similar cell density in control and speared groups up to about 72 hours. FIG. 11A and FIG. 11B show nuclei staining with DAPI in cortex neurons 72 hours after spreading. FIG. 11A shows the control cells; FIG. 11B shows the speared cells. These results suggest that nanotube spearing can generate perturbation to cells, but it is minor enough for neurons to recover. The exogenous gene was expressed after the recovery in about 48 hours.

### Discussion:

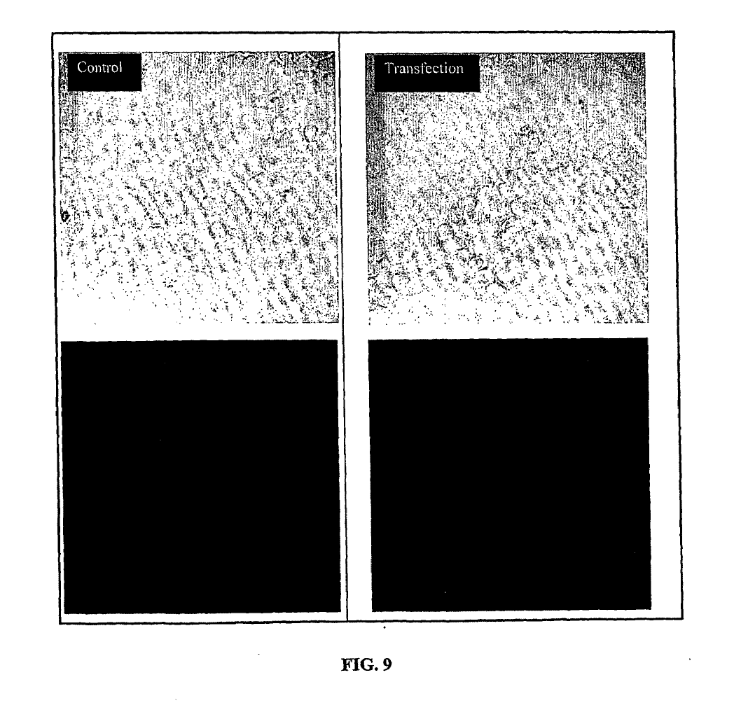

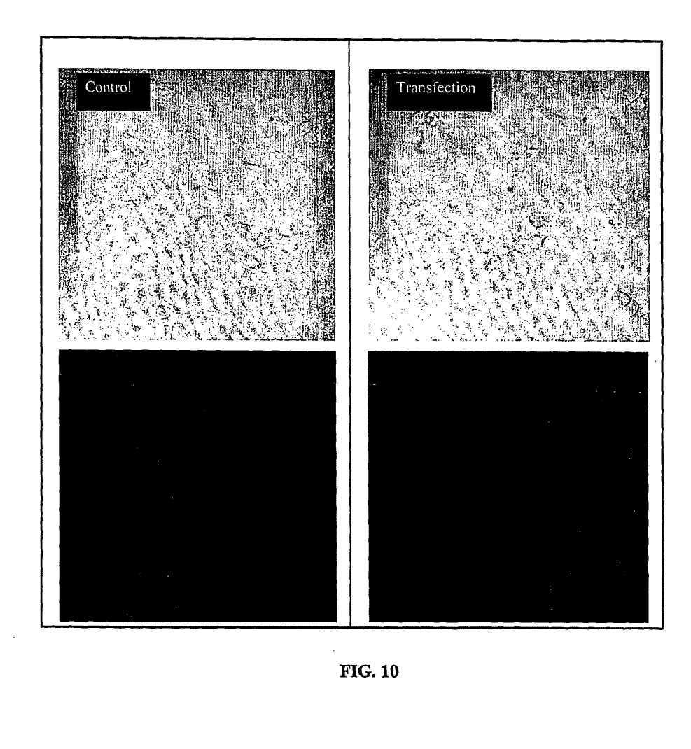

The results of the above-discussed experiments demonstrate the highly efficient molecular delivery of plasmid DNA into ex vivo neurons and splenic B cells, and transformed murine B lymphocytes by nanotube spearing technique. In contrast, EGFP expression was not detectable using Lipofectamine 2000 as a vehicle for plasmid transduction in both Bal17 cells and ex vivo splenic B cells. To illustrate this point, FIG. 9 shows EGFP transfection with Lipofectamine 2000 in Bal17 cells. The left panel of FIG. 9 shows a control; the right panel of FIG. 9 shows Bal17 cells which have been transfected. FIG. 10 shows EGFP transfection with Lipofectamine 2000 in primary B cells. The left panel of FIG. 10 shows a control; the right panel of FIG. 10 shows primary B cells which have been transfected. The high efficiency of transduction with nanotube spearing results from the unique delivery mechanism of the presently disclosed embodiments: nano-penetration of the cell membrane. As mentioned above, various transfection experiments were conducted at various conditions (see Table 1). Those results excluded the delivery through uptake pathways such as endocytosis and pinicytosis. Therefore, the results strongly suggest the occurrence of membrane penetration. Because of the nanoscale in this mechanical impact, the process only put a minor perturbation to cells as vulnerable as primary neurons; as a result, the viability of these cells remains unchanged after the spearing.

The expression of exogenous genes in B cells and neurons represent several challenges with the greatest being the low efficiency of transduction. The current results indicate that the presently disclosed embodiments of delivering macromolecules to cells by nanospearing may be particularly useful with low efficiency transduced cells. In fact, some proprietary products, such as Amaxa's Nucleofactor, were tested for non-viral transfection in primary cells. However, the efficiency has never reached as high as that observed by the nanotube spearing technique of the present method. Although the experiments outlined above focused on the delivery of plasmid DNA, the presently disclosed embodiments may be used with other macromolecules, such as proteins or peptides and RNAi conjugates, into mammalian cells exploiting the intensively studied surface chemistry for immobilizations.

According to recent studies, carbon nanotubes can facilitate the delivery of macromolecules in a variety of ways. It was demonstrated that carbon nanotubes can be internalized by an unidentified mechanism. Similarly, cellular uptake of carbon nanotubes by an endocytosis pathway has been observed, In both studies, the cells showed appreciable signals of the immobilized indicators after a period of incubation with about 1 μM-5 μM carbon nanotubes. In contrast to the above known processes, the method of the presently disclosed embodiments only needs as low as about 100 fM of nanotubes for almost ideal transduction efficiency. As such, the utilization of magnetic force results in 10⁷ times differences in the molecular shuttling efficiencies. The magnetic force mediated cell may fundamentally provide a convenient targetable gene delivery approach, in which cells were pinned on an array of nanotube bundles with attached plasmids.

Of note, the nanotube spearing technique of the presently disclosed embodiments can be optimized for better performance. In an embodiment, the DNA dosage is reduced for each transduction. As such, in one experiment, the DNA plasmid was used at the saturating concentration, which was about 10³ times higher than that of the nanotubes. Future quantitative assays of the nanotube functionalization and plasmids immobilization can help to reduce plasmids consumption without affecting the transducing effectiveness. Therefore, the process may be optimized from an economic point of view.

As such, the experiments show that using the nanotube spearing technique of the presently disclosed embodiments, various macromolecules have been successfully delivered into dividing and non-dividing cells. The latter, i.e. primary B cells and neurons, are notoriously hard-to-transfect. According to the above-discussed experimental results, nanotube spearing, as a non-viral approach, presented a transduction efficiency matching to that of the virals. Therefore, the method of the presently disclosed embodiments provides a great improvement in the field of macromolecule delivery into cells.

### Methods:

The following section describes the preparation of various components used in the experiments described above. This section is in no way meant to limit the scope of the presently disclosed embodiments. As such, those skilled in the art will recognize that various materials and methods of preparing those materials are within the spirit and scope of the presently disclosed embodiments.

### Nanotube Preparation

A Si wafer was coated with Cr and Ni layer of 350 and 30 nm, respectively. The nanotubes were grown in a hot filament PECVD system. A base pressure of 10⁻⁶ Torr was used prior to the introduction of acetylene and ammonia gases. The growth pressure was about 10-20 Torr, and the growth time was about 1-10 minutes for proper nanotube length control. The substrate temperature was maintained below about 660° C. The nanotubes were then scrapped-off from the 2×2 cm² Si wafer, and finally suspended in 5 ml ethyl alcohol with an estimated concentration of about 1 pM.

The suspension was centrifuged at about 10,000 rpm for about 10 minutes and the nanotubes were then separated out and suspended in about 0.5M HNO₃ that is too dilute to break well enclose graphene layer. The container was placed beside a Nd—Fe—B magnet overnight. The nanotubes attached to sidewall next to the magnet were collected and washed three times with de-ionized water by repeating the centrifuge and suspending cycle. The nanotubes were end up in about 5 ml ethyl alcohol as stock.

The nanotubes extracted from 1 ml stock were mixed with 5 μg plasmid and 10 mg EDC (1-ethyl-3-(3-dimethylaminopropyl) carbodiimide) in 0.1M MES buffer (2-[N-morpholino] ethane sulfonic acid at pH 4.5) for the aminization between the primary amine groups in the DNA molecules and carboxylic groups on carbon nanotubes. The reaction mixture was left in the dark at room temperature for about 1 hour. Finally, the nanotubes were precipitated by centrifugation and resuspended in about 1 ml serum free culture medium immediately before use.

### Primary Cell Preparations

Balb/c mice were purchased from The Jackson Laboratories (Bar Harbour, Me.) and housed at Boston College. Mice were cared for and handled at all times in accordance with National Institutes of Health and institutional guidelines. Splenic B cells were purified by depletion of T cells with anti-Thy-1.2 plus rabbit complement; macrophages (and other adherent cells) were removed by plastic adherence. Red blood cells and non-viable cells were removed by sedimentation on Lympholyte M gradients. The resulting B cells were cultured in RPMI-1640 medium (Sigma-Aldrich) supplemented with 10 mM Hepes, pH 7.5, 2 mM L-glutamine, 5×10⁻⁵ M 2-ME, 100 U/ml penicillin, 100 μg/ml strptomycin, 0.25 μg/ml fungizone, and 10% heat inactivated FCS (Atlanta Biologicals).

Mouse cortical neurons were extracted from E14 embryonic C57/BL6 mouse. The dissection was conducted under tissue culture hood. The cortices was separated, washed, cut in PBS, then digested with trypsin and Dnase. The cells were disassociated by pipetting up and down, resuspended in Neurobasal Medium (Invitrogen) that contained B27, 1% penicillin G and streptomycin and 2 μM glutamine, and plated on poly-D-lysine coated coverslips at the density of 1×10⁵ cells/cm². The cortical neuron cultures were treated with about 10 μg AraC to remove glial cells at day 3 or day 4 after plating. The cells were subjected to nanotube spearing after 24 hours of AraC treatment.

### Spearing

The cells were dispersed on poly-D-lysine coated substrates, such as grids and cover slips. A beaker containing about 10 ml serum free medium supplemented with nanotubes (0.1 pM), was placed on a magnetic stirrer (Fisher Scientific) at room temperature. The cover slips were picked up with a tweezer and vertically placed in Dulbecco's Modified Eagle Medium (Invitrogen) with the cells facing the incoming nanotubes. The speed of the stirrer was set at about 1200 rpm.

To enhance the nanotube spearing with the static field, the cell dish was laid on a Nd—Fe—B permanent magnet. An adaptor was sandwiched between the dish and magnet with groves machined on the surface to produce a stray field with high gradients in their close vicinity to improve the magnetic force.

### Electron Microscopy

Scanning electron microscopic (SEM) imaging was conducted using JEOL JSA-6340F. Before SEM imaging, MCF-7 cells on grid were fixed in a phosphate buffered saline (PBS) containing about 4% paraformaldehyde, then dehydrated in ethanol solutions of sequential concentrations: 50, 70, 95, and 100% (v/v in H₂O). After drying in air at room temperature, the sample grid was deposited with a gold film of 5 nm thick.

All patents, patent applications, and published references cited herein are hereby incorporated by reference in their entirety. While the presently disclosed embodiments have been particularly shown and described with references to preferred embodiments thereof, it will be understood by those skilled in the art that various changes in form and details may be made therein without departing from the scope of the presently disclosed embodiments encompassed by the appended claims.

1. A method of delivering a macromolecule to a B cell comprising:
engaging a macromolecule to at least one nanotube wherein the nanotube comprises a magnetic particle;
placing the nanotubes into a sample wherein the sample comprises a plurality of cells;
applying a force to the sample wherein the force causes the nanotubes to collide with the cells to spear the cells; and
applying a second force which drives the nanotubes into the cell.

2. The method of claim 1 wherein the cell is a Bal17 B-lymphoma cell.

3. The method of claim 1 wherein the cell is an ex vivo primary B cell.

4. The method of claim 1 wherein the cell is a primary cell.

5. The method of claim 1 wherein the cell is a human B cell.

6. The method of claim 1 wherein the macromolecule is DNA.

7. The method of claim 1 wherein the macromolecule is RNAi.

8. The method of claim 1 wherein the macromolecule is a protein.

9. The method of claim 1 wherein the macromolecule is a DNA plasmid.

10. The method of claim 9 wherein the DNA plasmid encodes for an enhanced green fluorescent protein.

11. A method of transfecting a B cell comprising:
engaging a macromolecule to at least one nanotube wherein the nanotube comprises a magnetic particle;
placing the nanotubes into a sample wherein the sample comprises a plurality of cells;
applying a force to the sample wherein the force causes the nanotubes to collide with the cells to spear the cells;
applying a second force which drives the nanotubes into the cells; and
disengaging the macromolecule from the nanotube.

12. The method of claim 11 wherein the magnetic particle comprises nickel.

13. The method of claim 11 wherein the first force is generated by a magnet.

14. The method of claim 11 wherein the cell is a Bal17 B-lymphoma cell.

15. The method of claim 11 wherein the cell is an ex vivo primary B cell.

16. The method of claim 11 wherein the cell is a primary cell.

17. The method of claim 11 wherein the macromolecule is DNA.

18. The method of claim 11 wherein the macromolecule is RNAi.

19. The method of claim 11 wherein the macromolecule is a protein.

20. A method of transducing a B cell comprising:
engaging a macromolecule to a nanotube wherein the nanotube comprises a magnetic particle;
placing the nanotubes into communication with a plurality of cells;
applying a force to the nanotubes which causes the nanotubes to collide with the cells and pierce the cellular membrane; and
disengaging the macromolecule from the nanotube.

21. (canceled)

22. (canceled)

23. (canceled)

24. (canceled)

25. (canceled)

26. (canceled)

27. (canceled)

28. (canceled)

29. (canceled)
